(19) United States
(12) Patent Application Publication
(10) Pub. No.: US 2014/0207000 A1
(43) Pub. Date: Jul. 24, 2014

(54) COATING FOR IMPROVING THE ULTRASOUND VISIBILITY

(71) Applicants: Dennis Manuel Vriezema, AJ Nijmegen (NL); Lee Ayres, AJ Nijmegen (NL); Abraham Reiner Keereweer, Grave (NL)

(72) Inventors: Dennis Manuel Vriezema, AJ Nijmegen (NL); Lee Ayres, AJ Nijmegen (NL); Abraham Reiner Keereweer, Grave (NL)

(73) Assignee: ENCAPSON B.V., AJ Nijmegen (NL)

(21) Appl. No.: 14/114,177

(22) PCT Filed: Apr. 25, 2012
(86) PCT No.: PCT/NL12/50276

§ 371 Date: Jan. 13, 2014

(30) Foreign Application Priority Data
Apr. 26, 2011 (NL) 2006665

(51) Int. Cl. A61B 8/08

(52) U.S. Cl. CPC A61B 8/481; A61B 8/0841
USPC 600/458; 427/2.12; 523/105

(57) ABSTRACT

The disclosure relates to a coating for improving the ultrasound visibility of a device, the coating being made of a matrix material comprising at least one contrast agent, wherein the at least one contrast agent is a plurality of gas-filled microparticles. Moreover, this disclosure relates to an ultrasonic contrast agent-containing coating for a device. In addition, the herein-described disclosure relates to a method for preparing a microparticle, a method for preparing a coating, and a method for coating a device as well as the coated device.

### CROSS-REFERENCE TO RELATED APPLICATIONS

This application is a national phase entry under 35 U.S.C. §371 of International Patent Application PCT/NL2012/050276, filed Apr. 25, 2012, designating the United States of America and published in English as International Patent Publication WO 2012/148265 A1 on Nov. 1, 2012, which claims the benefit under Article 8 of the Patent Cooperation Treaty and under 35 U.S.C. §119(e) to The Netherlands Patent Application Serial No. 206665, filed Apr. 26, 2011.

### TECHNICAL FIELD

The disclosure relates to a coating for improving the ultrasound visibility of a device. In addition, this disclosure relates to an ultrasonic contrast agent-containing coating for a device. This disclosure also relates to a method for preparing a microparticle for incorporation in a coating for a device. Moreover, the herein-described disclosure relates to a method for preparing a coating. In addition, the disclosure relates to a device provided with an inventive coating, as well as a method for coating a device, and the use of a coating disclosed herein. The device is preferably a medical device, but is not limited thereto.

### BACKGROUND

Ultrasound is commonly used for the visualization of medical devices inside patients. It has been used to guide needles and catheters, for guide wire placement by radiologists, and by anesthesiologists for vascular access, nerve blockade, drainage of pleural or ascetic fluid collections and percutaneous tracheotomy. With ultrasound, it is possible to identify the target and collateral structures and real-time guidance to precisely place needles. The advantage of ultrasound, when compared to X-ray imaging, is that the energy of the sound waves is sufficiently low so as not to harm the patient.

Diagnostic sonography (ultrasonography) is an ultrasound-based diagnostic imaging technique used for visualizing subcutaneous body structures including tendons, muscles, joints, vessels and internal organs for possible pathology or lesions. Sonography (ultrasonography) is widely used in medicine. It is possible to perform both diagnosis and therapeutic procedures, using ultrasound to guide interventional procedures (for instance, biopsies or drainage of fluid collections). Sonographers are medical professionals who perform scans for diagnostic purposes. Sonographers typically use a hand-held probe (called a transducer) that is placed directly on and moved over the patient. The creation of an image from sound is done in three steps—producing a sound wave, receiving echoes, and interpreting those echoes. The sound wave is partially reflected from the layers between different tissues. Specifically, sound is reflected anywhere there are density changes in the body, e.g., blood cells in blood plasma, small structures in organs, etc. Some of the reflections return to the transducer. The return of the sound wave to the transducer results in the same process that is used to send the sound wave, except in reverse. The return sound wave vibrates the transducer and the transducer turns the vibrations into electrical pulses that travel to the ultrasonic scanner where they are processed and transformed into a digital image. The use of micro-bubble contrast media in medical sonography to improve ultrasound signal backscatter is known as contrast-enhanced ultrasound.

As stated, reflection of a sound wave occurs when it strikes the boundary between two media. Depending upon the angle of reflection, sound waves can return to the transducer probe to provide a signal. Optimum echoes would be provided by ultrasound waves that are reflected back at 90°, but this will only be the case for some reflected sound waves. Reflection at other angles may result in a distorted image, artefact or loss of signal. Needle visualization is essential when inserting needles into tissues, which may be in close proximity to structures such as vessels, nerves and the pleura. Without accurate identification of the position of the needle, it is possible that damage to collateral structures may occur. Subsequent visualization of catheters and guide wires within target structures also promotes safe practice and minimizes discomfort for the patient.

Echo is the reflection of sound and echogenicity is the ability to bounce an echo, i.e., return the signal in ultrasound examinations. In other words, echogenicity is higher when the surface bouncing the sound echo reflects more sound waves.

Several methods have been described to improve the echogenicity of needles and other medical devices.

U.S. Pat. No. 4,401,124 discloses a biopsy needle showing a diffraction grating by a multiplicity of grooves. The modifications to the surface of the needle will possibly result in a weakening of the mechanical properties of the needle.

U.S. Pat. No. 5,289,831 A discloses medical devices that have an acoustic impedance that is different from the surrounding medium. This effect is, e.g., created by the presence of a plurality of partially spherical indentations on the surface. Also, the possibility of glass particles is mentioned, but they do not give as much echogenicity as hollow, gas-filled silicate microspheres.

WO 98/19713 discloses a highly porous coating-containing gas, entrapped in enclosed bubbles or open surface channels or cavities in a matrix. This is difficult to produce and will possibly have a lack of mechanical stability. There is no mention of adding discrete gas-filled hollow microparticles to a coating in order to create an echogenic device.

U.S. Patent Publication 2009/177114 A1 discloses a needle with a non-circular transverse crosssection over at least a portion of its length. A drawback of this method is that the shape of the needle is no longer spherical, which may alter the properties of the needle in terms of penetration of the skin and tissue and it will not be as flexible as a spherical needle.

WO 2008/148165 A1 discloses a medical device with enhanced ultrasonic reflectivity with at least one indentation over only part of its periphery created by surface modification. The medical device may have weakened mechanic properties.

WO 2007/089761 discloses lubricious echogenic coatings containing a plurality of microparticles. A disadvantage of this method is that multiple layers have to be applied for this to be effective.

WO 2010/059408 A2 discloses a medical device having coated tungsten and tungsten carbide particles, which is a costly process.

WO 00/51136 A1 discloses a medical device having enhanced ultrasound visibility because of a coating comprising a matrix material containing a plurality of contrast-enhancing elements. As examples of contrast-enhancing elements, reference is made of U.S. Pat. Nos. 5,289,831 A and 5,081,997 (of the same applicant) and to U.S. Pat. Nos. 5,741,522 and 5,776,496 (both of the same applicant). U.S. Pat. Nos. 5,741,522 and 5,776,496 relate to non-aggregated porous particles of uniform size for entrapping gas bubbles within. These non-aggregated porous particles are very different from the microparticles disclosed herein, e.g., since they are not used to enhance the ultrasound visibility of devices. In U.S. Pat. No. 5,648,095 (referenced in WO 00/51136 A1), methods are described to prepare hollow microparticles as contrast agents for ultrasound. These hollow microparticles of U.S. Pat. No. 5,648,095 are not incorporated in a coating matrix to enhance the ultrasound visibility of devices. Moreover, U.S. Pat. No. 5,648,095 discloses different types of polymers for forming the microparticles than disclosed herein.

The ultrasound-enhancing particles described in EP-A 0,500,023 (referenced in WO 00/51136 A1) are solid clay particles, which are different than the particles disclosed herein. The solid clay particles are not mixed in a matrix material.

EP 1 118 337 A2 discloses medical devices coated with an echogenic material that includes an electrically insulative base layer and an echogenic layer formed on the base layer. In EP 1 118 337, the particles are preferably small glass microspheres. However, there is no disclosure of discrete gas-filled hollow microparticles as contrast agents.

EP 0 624 342 A1 discloses a medical instrument with selected locations along the instrument that are provided with deposits of echogenic material. This material is preferably a polymeric foam having a matrix of gas bubbles contained therein. The disadvantage of this method is that it is difficult to control the layer thickness and to vary the coating matrix.

WO 98/48783 A1 discloses microparticles that are useful as ultrasonic contrast agents and for delivery of drugs. There is no reference that these microparticles are used in a coating to make a medical device echogenic.

There is a need for providing medical devices with a better ultrasound visibility without the drawbacks of the prior art.

The object of the disclosure is to improve the ultrasound visibility or echogenicity of a device and allow real-time monitoring of the location and position of the device without the drawbacks of the prior art. There is also a need to be able to apply the coating in an efficient and reproducible manner and to have good flexibility in the coating matrix that is used, since each type of substrate requires its own coating matrix.

One or more of the objects of this disclosure are obtained by a coating for improving the ultrasound visibility of a device, the coating being made of a matrix material comprising at least one contrast agent, wherein the at least one contrast agent is a plurality of gas-filled microparticles.

By the application of a coating comprising gas-filled microparticles, the ultrasound visibility of the device is enhanced. When the device is a medical device that is inserted into a human or animal body and that is studied at angles that deviate from 90° with respect to the ultrasound transducer, the ultrasound visibility remains good. Due to the difference in acoustic impedance between the tissue surrounding the medical device and the gas bubbles trapped inside the microparticles, a much enhanced image of the medical device can be obtained during ultrasound visualization.

### DISCLOSURE

The disclosure includes the following embodiments. This list is non-limiting.

In one embodiment, at least 80% of the microparticles have curved surfaces.

In another embodiment, at least 90% of the microparticles have curved surfaces.

In yet another embodiment, at least 80% of the microparticles are substantially spherical.

In yet another embodiment, at least 90% of the microparticles are substantially spherical.

In yet another embodiment, at least 80% of the microparticles have a diameter in the range of 0.5 to 500 microns.

In yet another embodiment, at least 90% of the microparticles have a diameter in the range of 0.5 to 500 microns.

In yet another embodiment, the microparticles have a diameter in the range of 0.5 to 100 microns.

In yet another embodiment, the microparticles have a diameter in the range of 1 to 50 microns.

In yet another embodiment, the microparticles are in the form of a hollow center surrounded by a wall, wherein a gas is present within the hollow center.

In yet another embodiment, the wall has a wall thickness in the range of 0.2 to 20 micron.

In yet another embodiment, the wall has a wall thickness in the range of 1 to 5 micron.

In yet another embodiment, the density of the microparticles is between 10⁶/mm² and 1/mm².

In yet another embodiment, the density of the microparticles is between 10⁴/mm² and 400/mm².

In yet another embodiment, the gas with which the microparticles are filled is selected from the group consisting of air, nitrogen, oxygen, a noble gas, a fluorinated gas, or a hydrocarbon.

In yet another embodiment, the microparticles are made from a material selected from one or more of the group consisting of polymers, ceramics, glasses, organic materials, and metals and one or more combinations thereof.

In yet another embodiment, the matrix material is selected from the group of polymers. With “polymers” is meant polymeric and oligomeric structures having at least ten repeating monomer units. A polymer is a large molecule (macromolecule) composed of repeating structural units. These subunits are typically connected by covalent or non-covalent chemical bonds.

In yet another embodiment, the polymer is selected from the group consisting of a poly(ether sulfone), a polyisocyanate, a polyurethane, a polytetrafluoroethylene, a polymer or copolymer of N-vinyl-pyrrolidone, a poly(4-vinyl pyridine), a polyacrylamide, a poly(amido-amine), a poly(ethylene imine), a block copolymer of ethylene oxide and propylene oxide, a block copolymer of styrene, a polydialkylsiloxane, a polysaccharide, a polystyrene, a polyacrylate, a polyalkane, a poly(ether ketone), a polyester, a polyamide, a polyalkylmethacrylate, and one or more combinations thereof.

Preferably, the polymer for the matrix material is selected from poly(ether sulfones), polyurethanes, polyacrylates, polymethacrylates, and one or more combinations thereof.

In yet another embodiment, at least one additional contrast agent, preferably an MRI and/or an X-ray contrast agent, is present in the matrix material of the coating.

In yet another embodiment, the ratio of the microparticles to the matrix material in the coating is between 0.01 to 50 wt. %, more preferably, a ratio of 0.1 to 20 wt. %.

Moreover, the herein-described disclosure relates to an ultrasonic contrast agent-containing coating for a device, the coating being made of a matrix material comprising at least one contrast agent, wherein the at least one contrast agent is a plurality of gas-filled microparticles.

In addition, this disclosure relates to a method for preparing a microparticle, comprising the steps of:
i) providing the shell-forming material;
ii) dissolving the shell-forming material and a non-volatile liquid in a volatile, non-water-miscible solvent;
iii) introducing the solution of step ii) to a stirred aqueous solution containing a surfactant (e.g., poly(vinyl alcohol));
iv) removing the volatile, non-water-miscible solvent from the mixture of step iii) (e.g., by evaporation under reduced pressure, solvent extraction or continued stirring until all solvent has evaporated;
v) concentrating the microparticles formed in step iv) by filtration and washing off of the surfactant with water; and
vi) drying of the microparticles (e.g., by freeze-drying).

In addition, the disclosure relates to a method for preparing a coating, the method comprising the steps of:
1) providing at least one contrast agent in the form of a plurality of gas-filled microparticles;
2) providing a matrix material; and
3) combining the matrix material of step 2) with the at least one contrast agent of step 1) to form the coating.

In addition, this disclosure relates to an inventive coating or a device prepared according to the disclosed method or a device as provided with a coating for improving the ultrasound visibility thereof.

A further embodiment of the device disclosed herein relates to a medical device, preferably selected from the group consisting of a needle, a biopsy needle, a cannula, a catheter, a feeding tube, a forceps, an introducer, a tissue marker, a stylet, a guide wire, a stent, a vascular dilator, a biopsy site marker, a retrieval snare, an angioplasty device, a tube, an implanted cardiac resynchronization device and a trocar.

Moreover, the herein-described disclosure relates to a method for coating a device, this method comprising the steps of:
A) providing a device;
B) providing an inventive coating; and
C) applying the coating of step B) to the device of step A).

In an embodiment of this method, the gas-filled microparticles are incorporated in the matrix material during the manufacturing of the device.

In yet another embodiment of this method, the coating can be applied to the device via coating methods such as dip-coating, spray-coating, stamping, inkjet printing and drop-casting.

In another embodiment hereof, the use of a coating is provided for coating a device in order to improve the ultrasound visibility thereof.

### BRIEF DESCRIPTION OF THE DRAWINGS

The disclosure is furthermore illustrated by the following drawings:

FIG. 1 discloses a schematic overview of a tip of a needle provided with a coating disclosed herein;

FIG. 2A (needles in phantom) discloses a sonographic picture of a needle that is not coated and FIG. 2B discloses a sonographic picture of a needle that is coated as disclosed herein;

FIG. 3 discloses a graph comparing the acoustics of a coated and uncoated metal wire;

FIG. 4 shows a graph of the echogenicity of coated needles;

FIG. 5 shows a graph of the echogenicity in water of plastic tubes coated with gas-filled silicate microspheres; and

FIG. 6A shows a sonograph of uncoated plastic tube and FIG. 6B shows a sonograph of coated plastic tube.

### DETAILED DESCRIPTION

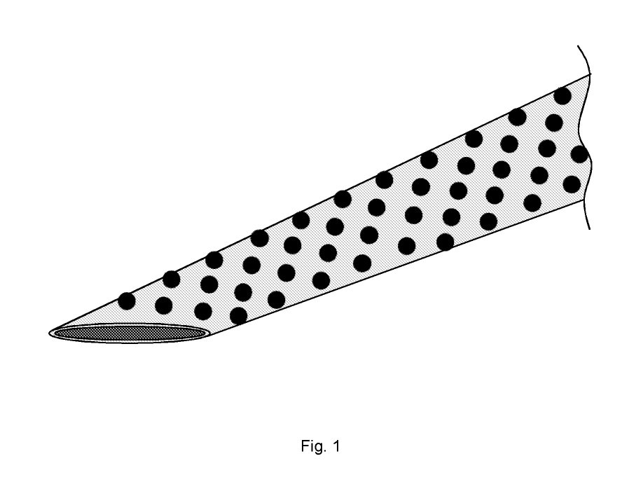

As disclosed above, the coating disclosed herein increases the echogenicity of a device that is provided with the inventive coating. FIG. 1 shows a schematic overview of a device, in this case a tip of a needle, that is provided with a coating as disclosed herein. The coating is disclosed in grey scale and the gas-filled microparticles (microspheres not drawn to size) are shown in the tip of the needle. It should be noted that the gas-filled microparticles as disclosed herein can be fully embedded in the present matrix material of the coating so that the outside surface of the device coated with the inventive coating has an almost completely smooth surface. FIG. 1 is merely schematic to show that the gas-filled microparticles can be distributed over a larger part or the totality of the device. It is also possible that the gas-filled microparticles are present on the outside surface of the coated device, in which case, a non-smooth surface or rough surface will be obtained. In addition, it should be noted that it is also possible to apply an additional coating or top layer, or more than one, on top of the inventive coating in order to further improve the required properties of the needle, such as a smooth surface, lubricity, or other properties.

Several embodiments of the disclosure will be explained in more detail below.

In an embodiment, at least 80%, preferably at least 90% of the microparticles have curved surfaces. Preferably, at least 80%, and more preferably, at least 90% of the microparticles are substantially spherical.

As a result of the curved surface, or even more, the spherical nature of the microparticles, the ultrasound waves are reflected in many directions, thus allowing the transducer to detect the device even when the angle between the surface of the device and transducer is much smaller than 90°. The gas-containing microparticles are preferably spherical. In that way, the sound waves are reflected in all directions.

With “having curved surfaces” is meant in this application that the surfaces of the microparticles primarily have curved instead of flat surfaces. Hence, the majority of the microparticles are not to be cubes, pyramids, etc. With “substantially spherical” is also meant elliptical or ellipsoidal, oval-shaped, oblong, oviform, ovoid, or egg-shaped, globular or ball-shaped. The specific form can be selected according to the criteria for production and use of the microparticles.

In an embodiment, at least 80%, preferably at least 90% of the microparticles have a diameter in a range of 0.5 to 500 microns. In a more preferred embodiment, the range of diameters of the microparticles is 0.5 to 100 microns, 1 to 50 microns.

It is possible to prepare several batches of microparticles having different sizes by several sieving actions. For example, the microparticles can be sieved through a sieve having a cut-off of 25 microns, hence, producing a batch that passes through the sieve (the filtrate) wherein at least 80%, preferably at least 90% of the microparticles have a diameter in the range of 0.5 to 25 microns. The retentate of the filtration step, hence, contains mostly nanoparticles having a size above 25 microns. This retentate may be subsequently passed through a sieve having a cut-off of 50 microns. The filtrate then is a batch of microparticles wherein at least 80%, preferably at least 90% of the microparticles have a diameter in the range of 25 to 50 microns. The retentate then comprises a microparticle batch wherein at least 80%, preferably at least 90% of the microparticles have a diameter in the range of 50 to 100 microns.

The diameter that is mentioned in this disclosure is the average thickness in all directions of the particles. For example, in case of an oval microparticle having a width, height and depth, the diameter as mentioned in this disclosure is the average of these three values. The ratio of the largest value of the width, height and depth over the smallest value for the width, height and depth is preferably between 1:4 and 4:1, more preferably between 1:3 and 3:1, even more preferably between 1:2 and 2:1, even most preferably between 1.0:1.5 and 1.5:1.0. In case of spherical (i.e., a ball-shaped or round) microparticles, the width, height and depth are all equal and the diameter is this value and the ratio mentioned above is 1.

In a further embodiment, the microparticles are in the form of a hollow center surrounded by a wall, wherein a gas is present within the hollow center. The presence of a hollow center within the microparticles allows for the presence of a gas bubble inside the microparticles for improving echogenicity.

In another embodiment, the wall preferably has a wall thickness in the range of 0.2 to 20 microns, more preferably 1 to 5 microns.

For example, microparticles having a diameter of approximately 20 microns and having a wall thickness of approximately 4 microns have a hollow center with a diameter of approximately 12 microns. Microparticles having a hollow center are also referred to as microcapsules in the present application.

In another preferred embodiment, the gas with which the microparticles are filled is selected from the group consisting of air, nitrogen, oxygen, a noble gas, a fluorinated gas, or a hydrocarbon and one or more combinations thereof. This gas is present within the hollow center of the microparticles or capsules.

In this application, with a “noble gas” is meant a gas selected from the group of helium (He), neon (Ne), argon (Ar), krypton (Kr), xenon (Xe), and radon (Rn). In this application, with a “fluorinated gas” is meant an alkane gas wherein part or all of the hydrogen atoms have been substituted by fluorine atoms, e.g., octafluoropropane. Examples of hydrocarbon gases are alkanes, alkenes and alkynes, such as methane, ethane, ethene, ethyne, propane, propene, and propyne.

The microparticles can consist of any material that can contain a gas in its interior and that can form the walls of the microparticle. The microparticles are preferably made from a material selected from one or more of the groups consisting of polymers, ceramics, glasses, organic materials, and metals and one or more combinations thereof.

Examples of ceramics that can be used are clays, quartz, feldspar, alumina, beryllia, ceria, zirconia, carbide, boride, nitride, silicide and composites thereof. Examples of glasses that can be used are quartz and silicate, with or without additives. Examples of organic material that can be used are saccharides, phospholipids, lipids, peptides, and proteins.

The coating is made of a matrix material, which serves as a basis for embedding the gas-filled or gas-containing microparticles. In other words, the gas-containing microparticles are embedded within a matrix material. Preferably, the matrix material is selected from the group of polymers. With “polymers” is meant polymeric and oligomeric structures having at least ten repeating monomer units. A polymer is a large molecule (macromolecule) composed of repeating structural units. These subunits are typically connected by covalent or non-covalent chemical bonds.

In a preferred embodiment, the polymer of the matrix material is selected from the group consisting of:
a poly(ether sulfone);
a polyisocyanate;
a polyurethane;
a polytetrafluoroethylene;
a polymer or copolymer of N-vinyl-pyrrolidone (e.g., a copolymer with butylacrylate);
a poly-(4-vinyl pyridine);
a polyacrylamide (e.g., a poly(N-isopropylacrylamide));
a poly(amido-amine);
a poly(ethylene imine);
a block copolymer of ethylene oxide and propylene oxide (e.g., a poly(ethylene oxide-block-propylene oxide) or a poly(ethylene oxide-block-propylene oxide-block-ethylene oxide));
a block copolymer of styrene (e.g., a poly(styrene-block-isobutylene-block-styrene) or poly(hydroxystyrene-block-isobutylene-block-hydroxystyrene));
a polydialkylsiloxane;
a polysaccharide;
a polystyrene;
a polyacrylate;
a polyalkane (e.g., polyethylene, polypropylene and polybutadiene);
a poly(ether ketone) (e.g., poly(ether ketone) or poly(ether ether ketone));
a polyester (e.g., poly(ethylene terephthalate), polyglycolide, poly(trimethylene terephthalate) or poly(ethylene naphthalate), poly(lactic acid), polycapralatone, or poly(butylene terephthalate);
a polyamide (e.g., nylon-6,6, nylon-6, a polyphthalamide or a polyaramide);
a polyalkylmethacrylate (e.g., a polymethylmethacrylate or a poly(2-hydroxyethylmethacrylate)); or
one or more combinations of the above.

The polymer is preferably selected from poly(ether sulfones), polyurethanes, polyacrylates, polymethacrylates, and one or more combinations thereof.

In one embodiment, at least one additional contrast agent, preferably an MRI and/or X-ray contrast agent, is present in the matrix material of the coating in addition to the microparticles. These contrast agents are, for example, iopromide, gadolinium complexes, barium sulphate, and iron oxide nanoparticles. These contrast agents may be incorporated in the matrix along with the microcapsules. It is also possible that after application of the coating disclosed herein, an additional coating layer is applied comprising an additional contrast agent such as an MRI and/or X-ray contrast agent.

The presence of these additional contrast agents further improves the visibility of the device coating with the present coating if it is used in an MRI or X-ray in addition to ultrasound.

In another embodiment, the ratio of the microparticles to the matrix material of the coating is between 0.01 to 50 wt. %, more preferably, a ratio of 0.1 to 20 wt. %. This has been found by the present inventor to give a balance between good ultrasound visibility on the one hand and good coating and adhesion properties on the other hand.

In an embodiment of the disclosure, the coating is applied on almost the whole surface of the device that is to be inserted into the patient.

In another embodiment hereof, the coating is applied only on parts of the surface of the device.

In a further embodiment, the coating is applied in the form of bands or stripes with a specific distance between the two neighboring bands or stripes for the clinician in determining distances in the patient.

In a further embodiment, this disclosure relates to an ultrasonic contrast agent-containing coating, in other words, a coating for a device containing an ultrasonic contrast agent, the coating being made of a matrix material comprising at least one contrast agent, wherein the at least one contrast agent is a plurality of gas-filled microparticles.

In addition, this disclosure relates to an ultrasonic contrast agent containing coating for a device.

All of the embodiments described above for the coating for improving the ultrasound visibility of a device are also applicable to the other embodiments.

The disclosure relates to a method for preparing a microparticle, comprising the steps of:
i) providing the shell-forming material;
ii) dissolving the shell-forming material and a non-volatile liquid in a volatile, non-water-miscible solvent;
iii) introducing the solution of step ii) to a stirred aqueous solution containing a surfactant;
iv) removing the volatile, non-water-miscible solvent from the mixture of step iii);
v) concentrating the microparticles formed in step iv) by filtration and washing off of the surfactant with water; and
vi) drying of the microparticles.

In a first step, a shell-forming material is provided. Examples of the material for preparing the microparticles as given above include polymers, ceramics, glasses, organic materials and metals.

In a second step, this shell-forming material (viz. the material that the microparticle is made of) as well as a non-volatile liquid in a volatile, non-water-miscible solvent. Examples of the non-volatile liquid are decane, dodecane, cyclooctane, and cyclodecane. Examples of the volatile, non-water-miscible solvent are dichloromethane, chloroform, ethyl acetate, diethylether, diisopropylether, and alkanes, such as pentane, hexane, and heptane.

In a third step, the solution obtained in step ii) is introduced into a stirred aqueous solution containing a surfactant. An example of such a surfactant is polyvinylalcohol. Other surfactants are also applicable.

In the fourth step, the method includes removing the volatile, non-water-miscible solvent from the mixture of step iii). This step can, for example, be carried out by evaporation under reduced pressure, by solvent extraction or continued stirring until all the solvent has evaporated.

The method furthermore includes in step v) concentration of a microparticle formed in step iv), by filtration and by washing of the surfactant with water.

The last step vi) in this method is drying the microparticles, for example, by means of freeze drying.

All of the embodiments described above for the coating for improving the ultrasound visibility of a device are also applicable to this embodiment of a method for preparing a microparticle.

The microparticles can be prepared in several ways, such as inkjet printing, emulsification, microreactor technology, self-assembly, templating, e.g., layer-by-layer deposition, or in situ capsule formation. The preferred method for preparing the microparticles is emulsification. With this method, the microparticles are prepared by dissolving the shell-forming or wall-forming material in a volatile organic solvent as disclosed above. This solution is then added to a stirred aqueous phase to form a biphasic organic-aqueous solution. The resulting mixture is stirred until all volatile solvent has evaporated. Other methods to remove the volatile solvent are by extracting the mixture with isopropanol/water or by rotary evaporation under reduced pressure. To the organic phase, a non-volatile non-solvent can be added to allow the formation of a cavity during the precipitation of the shell-forming material upon evaporation of the volatile solvent. This non-volatile non-solvent can be removed by freeze-drying, thus resulting in gas-filled microparticles. The gas that is trapped inside the microparticles is the gas that is used to aerate the freeze-dryer after drying is completed, mostly air. The walls of the microcapsules are sufficiently permeable to allow gas to diffuse. Incorporation of the gas-filled microcapsules in the matrix is done by premixing the microcapsules with the matrix before application of this mixture on the substrate. The microcapsules can also be incorporated in a polymer matrix during extrusion of the polymer. In this way, the microparticles get distributed through the whole of the polymer matrix. If the matrix is extruded into a shrink tube, then it can be shrunk onto a cylindrical object by heating it.

Moreover, this disclosure relates to a method of preparing a coating disclosed herein, comprising the steps of:
1) providing at least one contrast agent in the form of a plurality of gas-filled microparticles;
2) providing a matrix material; and
3) combining the matrix material of step 2) with the at least one contrast agent of step 1) to form the coating.

In the first step, at least one contrast agent in the form of a plurality of gas-filled microparticles is provided. In a second step, a matrix material that can be selected according to the description above is provided and in a third step, the material on step 2) is combined with at least one contrast agent of step 1) to form the coating. It is also possible to incorporate the gas-filled microparticles only during the manufacturing of the device, for example, during extrusion or injection moulding. In another method, the coating is first prepared and then applied to the device in one or several ways such as, for example, dip-coating, spray-coating, stamping, inkjet printing and drop-casting. The microcapsules have to adhere well to the surface of the substrate, so they should be at least partially embedded. In an embodiment, 1 micron is used as a minimum microparticle diameter, in which case, a minimum coating thickness of 0.5 micron is required to fully embed the microparticle. A desired upper limit for the coating thickness is 200 microns, and a preferred range for the coating thickness is 0.5 to 50 microns. The particles are preferably homogeneously distributed throughout the coating in a direction perpendicular to the thickness of the coating along the surface of the coating. In other words, there is a uniform distribution of particles in the coating. In comparison with other methods according to the prior art, the present method is very easy and allows use of the inventive coating in a wide variety of devices.

Moreover, the disclosure relates to a device provided with a coating disclosed herein or a device provided with a coating prepared according to a herein-disclosed method for improving the ultrasound visibility thereof.

In an embodiment, the device is a medical device selected from the group consisting of a needle, a biopsy needle, a cannula, a catheter, a feeding tube, a forceps, an introducer, a tissue marker, a stylet, a guide wire, a stent, a vascular dilator, a biopsy site marker, a retrieval snare, an angioplasty device, a tube, an implanted cardiac resynchronization device and a trocar.

In a further embodiment, this disclosure relates to a method for coating a device, the method comprising the steps of:
A) providing a device;
B) providing a coating disclosed herein or a coating prepared according to a herein-disclosed method; and
C) applying the coating of step B) to the device of step A).

In addition, the disclosure relates to the use of a coating disclosed herein or prepared according to the method of the disclosure for coating a device in order to improve the ultrasound visibility thereof.

A comparison was made between gas-filled microspheres and solid microspheres as contrast-enhancing agents in a coating on a device. For this purpose, hollow, gas-filled microspheres and solid silica microparticles, both with a diameter of 25 microns and smaller, were coated on plastic tubes using the same ratio of microparticles to coating matrix. The echogenicity of these tubes was then measured in water. The outcome of this experiment was that the tubes coated with hollow, gas-filled microspheres showed a greatly improved higher echogenicity with respect to the solid microspheres.

In another experiment, the influence of the particle density on the echogenicity of the medical device was tested. For this purpose, different amounts of hollow, gas-filled microspheres were mixed through the coating matrix and applied on a plastic tube. The outcome of this experiment was that when the density of the hollow, gas-filled microspheres is as close as possible to a hexagonal packing, the echogenicity is as high as possible. At very low densities, the echogenic effect is comparable to an uncoated device. In the table below, an overview is given of the results.

| | Dilution | |
|---|---|---|
| Type of microparticle | coating* | Echogenicity^{§} |
| None | None | − |
| None | 1.5 | = |
| None | 3.0 | −− |
| Silica | None | − |
| Silica | 1.5 | −− |
| Silica | 3.0 | + |
| Hollow, gas-filled silicate microspheres | None | + |
| (25 μm < ø > 50 μm) | | |
| Hollow, gas-filled silicate microspheres | 1.5 | = |
| (25 μm < ø > 50 μm) | | |
| Hollow, gas-filled silicate microspheres | 3.0 | + |
| (25 μm < ø > 50 μm) | | |
| Hollow, gas-filled silicate microspheres | None | + |
| (ø < 25 μm) | | |
| Hollow, gas-filled silicate microspheres | 1.5 | = |
| (ø < 25 μm) | | |

*n-Butyl acetate was used to dilute the coating matrix.
^{§}The echogenicity was determined with the naked eye, studying sonographs of these samples taken in turkey breast.
−− very poor
− poor
= same as blank (the blank is a non-coated object)
+ good

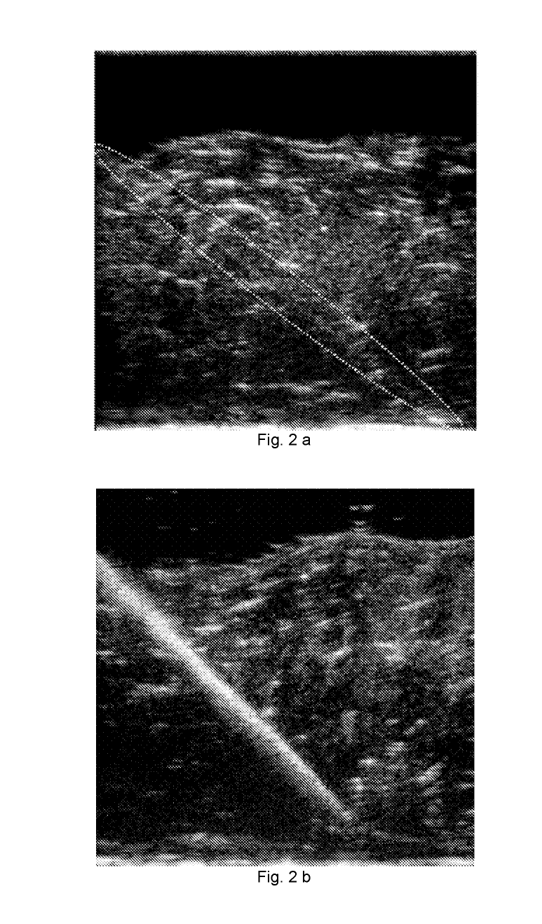

FIG. 2 shows two pictures taken during a sonographic experiment in a turkey breast. The needles were injected in the turkey breast and studied by an ultrasound transducer. FIG. 2a shows a picture of a needle within the turkey breast without a coating as disclosed herein. It is clear that the shape and length of the needle are only poorly visible. FIG. 2b shows a picture of a needle with a coating as disclosed herein with gas-filled microspheres according to Example 4 in a poly(ether sulfone) matrix. It is clearly visible that the needle shows an improved echogenicity and is clearly visible during the ultrasound experiment.

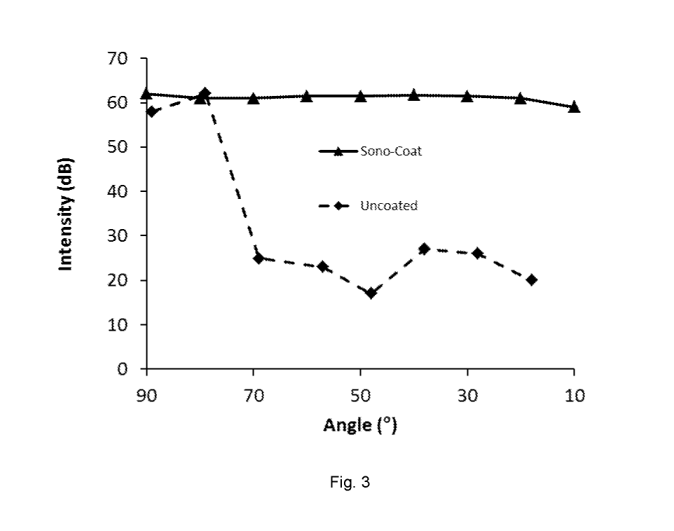

FIG. 3 shows a graph in which the acoustic signal as a function of the angle of the transducer with respect to the needle is plotted. It can be clearly seen that the uncoated needle is poorly visible at larger angles, whereas the needle coated with the coating disclosed herein (same coating as described for FIG. 2) remains highly visible. This experiment was carried out in water.

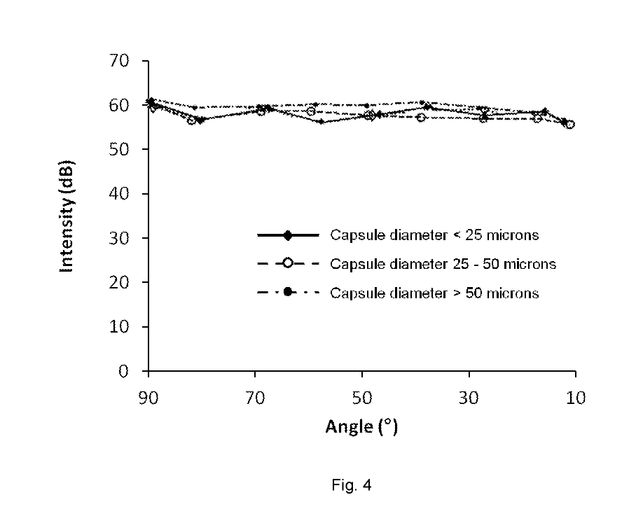

FIG. 4 shows a graph of the echogenicity of needles coated with gas-filled silicate microspheres according to Example 4 as a function of the angle between needle and transducer. It can be clearly seen that even at larger angles, the intensity remains high and that there is no difference in the echogenicity of the microspheres with different diameters.

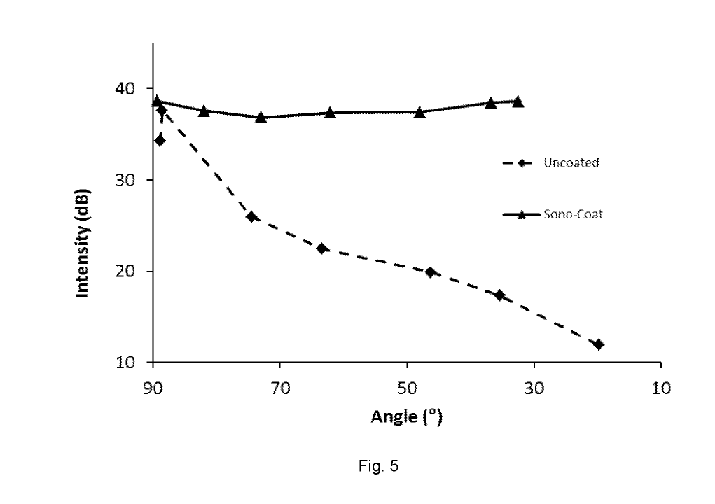

FIG. 5 shows a graph of the echogenicity in water of plastic tubes coated with gas-filled silicate microspheres according to Example 8 as a function of the angle between needle and transducer. It can be clearly seen that even at larger angles, the intensity remains high and that there is no difference in the echogenicity of the microspheres with different diameters.

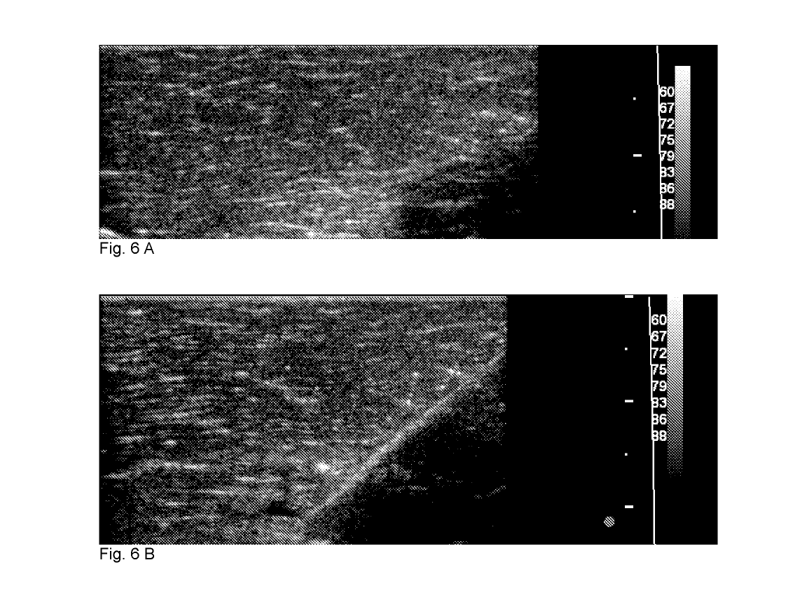

FIG. 6A shows sonographs of uncoated plastic tube and plastic tube coated according to Example 8 is shown in FIG. 6B.

This disclosure is now furthermore illustrated by several examples.

### EXAMPLES

### Example 1

To 100 ml of a stirred solution of 15 wt. % poly(N-vinyl-pyrrolidone)-co-poly(butyl acrylate) in ethanol, 5 g of gas-filled chitosan microcapsules were added. The microcapsules were prepared as published by S. Wang, D. M. Yu, J. Appl. Polym. Sci. 2010, 118:733, and were sieved using a sieve with a mesh size of 25 microns prior to addition. Into this mixture, cannulae were dip-coated and dried for two hours in an oven of 100° C.

### Example 2

To 100 ml of a stirred solution of 13 wt. % poly(ether sulfone) solution in N-methyl-pyrrolidone, 5 g of gas-filled chitosan microcapsules (as used in Example 1) were added. The microcapsules were sieved using a sieve with a mesh size of 25 microns prior to addition. Into this mixture, cannulae were dip-coated and dried for two hours in an oven of 100° C.

### Example 3

Gas-filled silicate microspheres were prepared as published by N. Xu, J. Dai, J. Tian, X. Ao, L. Shi, X. Huang, and Z. Zhu, Mater. Res. Bull., 2011, 46:92. The microsphere were sieved using a sieve with a mesh size of 25 microns and 5 g were then added to 100 ml of a poly(N-vinyl-pyrrolidone)-co-poly(butyl acrylate) solution (15 wt. %) in ethanol. The resulting coating was applied on a cannula by dip-coating and placed for two hours in an oven of 100° C.

### Example 4

Commercially available gas-filled silicate microspheres were sieved using a sieve with a mesh size of 25 microns and 5 g were then added to 100 ml of a poly(ether sulfone) solution (13 wt. %) in N-methyl-pyrrolidone. The resulting coating was applied on a cannula by dip-coating and placed for two hours in an oven of 100° C.

### Example 5

Gas-filled melamine-formaldehyde microcapsules were prepared as published by S. R. White, N. R. Sottos, P. H. Geubelle, J. S. Moore, M. R. Kessler, S. R. Sriram, E. N. Brown, and S. Viswanathan, Nature 2001, 409:794. These microcapsules were sieved using a sieve with a mesh size of 25 microns. A mixture of 5 grams of thus prepared microcapsules and 100 ml of 15 wt % poly(N-vinyl-pyrrolidone)-co-poly(butyl acrylate) in ethanol was prepared and cannulae were dip-coated into it and dried for two hours in an oven of 100° C.

### Example 6

Gas-filled melamine-formaldehyde microcapsules where prepared as disclosed in Example 5. A mixture of 5 g of microcapsules thus prepared, which were sieved using a sieve with a mesh size of 25 microns, and 100 ml of 13 wt. % poly(ether sulfone) in N-methyl-pyrrolidone was prepared and cannulae were dip-coated into it and dried for two hours in an oven of 100° C.

### Example 7

Gas-filled poly(methyl methacrylate) microcapsules were prepared as published by A. Loxly, B. Vincent, J. Colloid, Interface Sci. 1998, 208:49. A mixture of 5 g of microcapsules thus prepared, which were sieved using a sieve with a mesh size of 25 microns, and 100 ml of 15 wt. % poly(N-vinyl-pyrrolidone)-co-poly(butyl acrylate) in ethanol was prepared and cannulae were dip-coated into it and left to dry at room temperature.

### Example 8

Commercially available gas-filled silicate microspheres were sieved using a sieve with a mesh size of 50 microns followed by a sieve with a mesh size of 25 microns. The retentate in the sieve with a mesh size of 25 microns was used further. A mixture of 5 grams of the microspheres and 100 ml of a polyurethane coating (Labo coat of Labo Groep B.V.) was prepared. The resulting coating was applied on a polyurethane tube by dip-coating and left to dry at room temperature.

### Example 9

Different amounts of different microparticles were mixed through Labo coat. The same dipping process as described in Example 8 was used. In the table below, an overview is given of the ratio of microparticles to coating matrix and the type of microparticles. Furthermore, the table lists whether the Labo coat was used as is, or if it was diluted with n-butyl acetate.

| | Amount of | |
|---|---|---|
| | microparticles in | Dilution |
| Type of microparticle | coatng (wt. %) | coating |
| None | None | None |
| None | None | 1.5 |
| None | None | 3.0 |
| Silica | 0.95 | None |
| Silica | 1.00 | 1.5 |
| Silica | 0.99 | 3.0 |
| Hollow, gas-filled silicate microspheres | 1.00 | None |
| (25 μm < ø > 50 μm) | | |
| Hollow, gas-filled silicate microspheres | 0.96 | 1.5 |
| (25 μm < ø > 50 μm) | | |
| Hollow, gas-filled silicate microspheres | 0.99 | 3.0 |
| (25 μm < ø > 50 μm) | | |
| Hollow, gas-filled silicate microspheres | 0.98 | None |
| (ø < 25 μm) | | |
| Hollow, gas-filled silicate microspheres | 0.98 | 1.5 |
| (ø < 25 μm) | | |
| Hollow, gas-filled silicate microspheres | 1.01 | 3.0 |
| (ø < 25 μm) | | |

The most preferred embodiment is the embodiment having 1 wt. % of hollow, gas-filled glass microspheres with diameters between 25 and 50 microns in Labo Coat, which is diluted three times coated on the plastic tube.

### Example 10

Sterilization with gamma radiation and ethylene oxide (EtO) has been performed on medical devices coated according to Examples 4 and 8. The echogenicity of these devices was unchanged after sterilization.

The choice of coating material is dependent upon the surface that needs to be coated and the required properties, i.e., hydrophobic or hydrophilic. For metal surfaces, the poly(ether sulfone)-based coatings worked best in terms of adhesion and scratch resistance, whereas for plastic tubing, polyurethane coatings are preferred. The silicate microspheres were the most echogenic of all the tested microspheres. With a diameter below 25 microns, the coated cannulae and tubes felt smooth to the touch and the echogenicity was as high as for bare stainless steel wires with the transducer being perpendicular to the surface with respect to the wires (FIG. 4).

One or more of the objectives are obtained by this disclosure, embodiments of which are disclosed in the appended claims. The disclosure is not limited to the examples cited above.

1. Coating for improving the ultrasound visibility of a device, said coating being made of a matrix material comprising at least one contrast agent, characterized in that said at least one contrast agent is a plurality of gas-filled microparticles.

2. The coating of claim 1, wherein at least 80%, of the microparticles have curved surfaces.

3. The coating of claim 1, wherein at least 80%, of the microparticles have a diameter in the range of 0.5 to 500 microns.

4. The coating of claim 1, wherein the microparticles are in the form of a hollow centre surrounded by a wall, wherein a gas is present within the hollow center.

5. The coating of claim 1, wherein the gas with which the microparticles are filled is selected from the group consisting of air, nitrogen, oxygen, a noble gas, a fluorinated gas, or a hydrocarbon.

6. The coating of claim 1, wherein the microparticles are made from a material selected from one or more of the group consisting of polymers, ceramics, glasses, organic materials, and metals and one or more combinations thereof.

7. The coating of claim 1, wherein the matrix material is selected from the group consisting of polymers, polymer selected from the group consisting of a poly(ether sulfone); a polyisocyanate; a polyurethane; a polytetrafluoroethylene; a polymer or copolymer of N-vinyl-pyrrolidone such as a copolymer with butylacrylate; a poly(4-vinyl pyridine); a polyacrylamide such as poly(N-isopropylacrylamide); a poly(amido-amine); a poly(ethylene imine); a block copolymer of ethylene oxide and propylene oxide, a poly(ethylene oxide-block-propylene oxide), a poly(ethylene oxide-block-propylene oxide-block-ethylene oxide); a block copolymer, styrene, poly(styrene-block-isobutylene-block-styrene), poly(hydroxystyrene-block-isobutylene-block-hydroxystyrene); a polydialkylsiloxane; a polysaccharide; a polystyrene, a polyacrylate, a polyalkane such as polyethylene, polypropylene, polybutadiene, a poly(ether ketone), poly(ether ketone), poly(ether ether ketone), polyesters, poly(ethylene terephthalate), polyglycolide, Poly(trimethylene terephthalate), poly(ethylene naphthalate)), poly(lactic acid), polycapralatone, Poly(butylene terephthalate); polyamides, nylon-6,6, nylon-6, Polyphthalamides, polyaramides; a polyalkylmethacrylate, a polymethylmethacrylate, a poly(2-hydroxyethylmethacrylate) and combinations thereof.

8. The coating of claim 1, wherein at least one additional contrast agent is present in the matrix material of the coating.

9. The coating of claim 1, wherein the ratio of the microparticles to the matrix material in the coating is between 0.01 to 50 wt. %.

10. The coating of claim 1, wherein the density of the microparticles on the surface of a substrate is between 10⁶/mm² and 1/mm².

11. Ultrasonic contrast agent containing coating for a device, said coating being made of a matrix material comprising at least one contrast agent, characterized in that said at least one contrast agent is a plurality of gas-filled microparticles.

12. Method for preparing a microparticle, comprising the steps of:
i) providing a shell-forming material;
dissolving the shell-forming material and a non-volatile liquid in a volatile, non-water-miscible solvent;
iii) introducing the solution of step ii) to a stirred aqueous solution containing a surfactant;
iv) removing the volatile, non-water-miscible solvent from the mixture of step iii) by, for instance, evaporation under reduced pressure, solvent extraction or continued stirring until all solvent has evaporated;
v) concentrating the microparticles formed in step iv) by filtration and washing off of the surfactant with water; and
vi) drying of the microparticles by freeze-drying.

13. Method for preparing the coating of claim 1, said method comprising the steps of
a) providing at least one contrast agent in the form of a plurality of gas-filled microparticles;
b) providing a matrix material; and
combining the matrix material of step b) with the at least one contrast agent of step a) to form the coating.

14. Device provided with the coating claim 1.

15. A method for producing a device, said method comprising the steps of:
A) providing a device;
B) providing a coating according to claim 1;
C) applying the coating of step B) to the device of step A).

16. Method according to claim 15, wherein the gas-filled microparticles are incorporated in the matrix material during the manufacturing of the device, preferably coating is applied to the device via coating methods such as dip-coating, spray-coating, stamping inkjet printing and drop-casting.

17. A method of improving the ultrasound of a device, the method comprising:
coating the device with the coating according to claim 1 in order to improve the ultrasound visibility thereof.

18. The coating of claim 1, wherein at least 90% of the microparticles have curved surfaces.

19. The coating of claim 2, wherein the microparticles are substantially spherical.

20. The coating of claim 3, wherein at least 90% of the microparticles have a diameter in the range of 0.5 to 500 microns.

21. The coating of claim 20, wherein the microparticles have a diameter in the range of 0.5 to 100 microns.

22. The coating of claim 20, wherein at least 90% of the microparticles have a diameter in the range of 1.0 to 50 microns.

23. The coating of claim 1, wherein the microparticles are in the form of a hollow center surrounded by a wall, wherein the wall has a wall thickness in the range of 0.2 to 20 micron.

24. The coating of claim 1, wherein the matrix material is selected from the group poly(ether sulfones), polyurethanes, polyacrylates, polymethacrylates, and any combination thereof.

25. The coating of claim 8, wherein the at least one additional contrast agent comprises an MRI and/or an X-ray contrast agent.

26. The coating of claim 9, wherein the ratio of the microparticles to the matrix material in the coating is between 0.1 to 20 wt. %.

27. The coating of claim 1, wherein the density of the microparticles on the surface of a substrate is between 10⁴/mm² and 400/mm².

28. The device of claim 14, wherein the device is selected from the group consisting of a medical device, a needle, a biopsy needle, a cannula, a catheter, a feeding tube, a forceps, an introducer, a tissue marker, a stylet, a guide wire, a stent, a vascular dilator, a biopsy site marker, a retrieval snare, an angioplasty device, a tube, an implanted cardiac resynchronization device, and a trocar.
